(19) United States
(12) Patent Application Publication
(10) Pub. No.: US 2022/0054667 A1
(43) Pub. Date: Feb. 24, 2022

(54) INVISIBLE SINGLET FILM

(71) Applicants: University of Washington, Seattle, WA (US); Seattle Children's Hospital (dba Seattle Children's Research Institute), Seattle, WA (US)

(72) Inventors: James Chen, Seattle, WA (US); Tanner Clark, Seattle, WA (US); Thomas Lendvay, Seattle, WA (US)

(21) Appl. No.: 17/389,936

(22) Filed: Jul. 30, 2021

Related U.S. Application Data
(60) Provisional application No. 63/074,640, filed on Sep. 4, 2020.
(60) Provisional application No. 63/068,762, filed on Aug. 21, 2020.

(51) Int. Cl. A61L 2/00; A61L 2/08; A61L 2/025; A61K 41/00; A61K 9/70; A01N 25/34; A01P 1/00; A61N 5/06; A61N 7/00

(52) U.S. Cl. CPC A61L 2/0076; A61L 2/088; A61L 2/025; A61K 41/0057; A61K 41/0028; A61K 9/7007; A61N 2007/0004; A01P 1/00; A61N 5/062; A61N 5/0624; A61N 7/00; A61L 2202/26; A01N 25/34

(57) ABSTRACT

Antimicrobial films for generating singlet oxygen and their use is described. The antimicrobial films are generally thin films having two surfaces or faces. The first surface of the film is adhesive. Various adhesives, including, electrostatic charges, are possible. The second surface faces in a direction opposite from the first surface. The second surface of the film emits singlet oxygen when activated by light or ultrasound. Various photosensitizers can be incorporated onto the second surface of the films for generating the singlet oxygen. The singlet oxygen and other radical species generated from the antimicrobial films diffuses out from and in proximity to the films to form a layer or cloud of singlet oxygen at a concentration sufficient to inactivate microbial particles, e.g., viruses and other pathogens, that come within the singlet oxygen layer to provide a protective zone against microbial particles.

### CROSS-REFERENCE TO RELATED APPLICATION

This application claims the benefit of U.S. Provisional Application No. 63/074,640, filed on Sep. 4, 2020, and U.S. Provisional Application No. 63/068762, filed on Aug. 21, 2021, both applications are herein expressly incorporated by reference in their entirety.

### BACKGROUND

During viral pandemics such as the COVID-19 outbreak, one of the recommendations from governmental authorities and health officials is for the general public to wear a non-respirator type face mask. Face masks have been shown to afford a variable degree of protection for individuals in the vicinity of the face mask user, if the user is capable of spreading infected respiratory droplets and aerosols. Research has shown that a face mask worn by an infected individual is capable of trapping some of the user produced respiratory droplets. However, the trapping of droplets is incomplete, highly variable depending on the mask material, design, and fit on the user's face, and may not protect other individuals in the vicinity of the face mask user at all from user generated infected aerosols. Face masks can slip from correct positioning or be worn incorrectly. Some individuals may not tolerate wearing of a face mask due to pre-existing respiratory problems such as asthma, obstructive lung disease, or interfering facial hair. Face masks may not be tolerated in hot weather. Face masks can potentially cause acne, infections, and a variety of other skin injuries when worn tightly or for prolonged periods of time. Face masks can interfere with communication and are generally uncomfortable and are a difficult behavior adjustment for much of the public. Some members of the public will refuse to wear masks due to personal or religious reasons. Much research has also shown that face masks do little or nothing to protect the user from other individuals in the vicinity who may harbor viral infections due to insufficient barrier function and air leakage around the face mask perimeter, further disincentivizing general face mask use during viral outbreaks and pandemics. In addition, young children, others with certain disabilities, may not tolerate face masks, and there can be risks of accidental injury from mask straps when used by young children. Clearly, there is a need for alternatives to face masks that are more comfortable, less intrusive, more psychologically acceptable, and that function to protect the user in a way that typical facemasks do not.

### SUMMARY

An antimicrobial film includes a first surface facing in a first direction; a second surface facing in a second direction opposite from the first surface, wherein the first surface and the second surface extend along a width and a length of the film, and wherein a distance separating the first surface and the second surface corresponds to a thickness of the film; one or more material layers are included between the first and the second surfaces of the film; wherein the first surface of the film is adhesive; and wherein the second surface of the film emits singlet oxygen that is toxic to microbial particles incident on the second surface when activated by light or ultrasound.

In an example, the antimicrobial film comprises one or more photosensitizers in or on at least one material layer.

In an example, the one or more photosensitizers include one or more of erythrosine, riboflavin, or methylene blue.

In an example, the first surface includes electrostatic charges as an adhesive. In an example, the first surface is on a first material layer comprising polyvinylidene chloride, polyvinyl chloride, or polyethylene.

In an example, the antimicrobial film comprises a second material layer juxtaposed on the first material layer, wherein the second material layer includes a polymer, plastic, cellulose, and the second material layer is the second surface of the film.

In an example, the first side includes an adhesive coating as a first material layer.

In an example, the adhesive coating includes one or more adhesives selected from acrylates, silicones, and hydrocolloid substances.

In an example, the antimicrobial film comprises a polymer, plastic, or cellulose as a second material layer, the adhesive coating is juxtaposed on the second material layer.

In an example, the second material layer includes one or more photosensitizers, and the second material layer includes the second surface of the film.

In an example, more than one material layers between the first and second surfaces are non-separable.

In an example, the antimicrobial film comprises a peel layer juxtaposed on the first surface, the peel layer is separable from the film to expose the adhesive first surface.

In an example, the thickness of the film is from 1.0 micron to 10 mm.

In an example, the thickness of the film is from 1 mm to 10 mm.

In an example, the second surface comprises an absorbent material, and the one or more photosensitizers are absorbed in the absorbent material.

In an example, the absorbent material is rechargeable with the least one photosensitizer.

In an example, the antimicrobial film further comprises one or more portions that are transparent, translucent, or opaque to visible light.

A wearable object for the face or head includes a wearable object with means for holding the wearable object to the face or head; and the antimicrobial film is adhered to the wearable object.

In an example, the wearable object is a hat or glasses.

A method of inactivating pathogens in an oral cavity includes applying the antimicrobial film to a surface inside of an oral cavity; and applying light or ultrasound energy directed at the second surface of the antimicrobial film to cause the emission of singlet oxygen from the film in the oral cavity.

A film includes a first surface facing in a first direction; a second surface facing in a second direction opposite from the first surface, wherein the first surface and the second surface extend along a width and a length of the film, and wherein a distance separating the first surface and the second surface corresponds to a thickness of the film; one or more photosensitizers that are arranged to emit localized singlet oxygen that is toxic to microbial particles incident on the second surface, wherein the one or more photosensitizers are activated by one or more of light or ultrasound to generate the emitted singlet oxygen.

In an example, the film further comprises one or more material layers that are included between the first and second surfaces of the film, wherein the one or more material layers include the one or more photosensitizers.

In an example, the first surface of the film is adhesive.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of an antimicrobial film according to one embodiment;

FIG. 2 is a diagrammatical illustration of an antimicrobial film according to one embodiment;

FIG. 3 is a diagrammatical illustration of one example application of the antimicrobial films;

FIG. 4 is a diagrammatical illustration of one example application of the antimicrobial films;

FIG. 5 is a diagrammatical illustration of one example application of the antimicrobial films;

FIG. 6 is a diagrammatical illustration of one example application of the antimicrobial films;

FIG. 7 is a diagrammatical illustration of one example application of the antimicrobial films; and

FIG. 8 is a diagrammatical illustration of one example application of the antimicrobial films.

### DETAILED DESCRIPTION

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to any measurements “about” means +/−5%.

It shall be understood that the term “microbial”, as used herein refers to an infectious microorganism, pathogen, or agent, including one or more of a virus, viroid, bacterium, archaea, protists, protozoan, prion, fungus, toxin, or the like.

Also, it shall be understood that the term “immunogen”, as used herein refers to an antigen or any other substance that induces both an immune response by a patient's immune system and generation of antibodies that bind to the immunogen.

Generally, disclosed are antimicrobial thin films having an adhesive surface or face and an opposite facing surface or face that generates singlet oxygen via the absorption of light or ultrasound energy. Singlet oxygen and other radical species that are generated diffuses out from and in proximity to the thin films to form a layer or cloud of singlet oxygen at a concentration sufficient to inactivate microbial particles that come within the singlet oxygen layer. The singlet oxygen can form a zone of protection from various types of microbial particles in proximity to the antimicrobial films. The antimicrobial films can have wide application for use in prevention of infectious diseases and for the disinfection of objects and surfaces in a variety of settings.

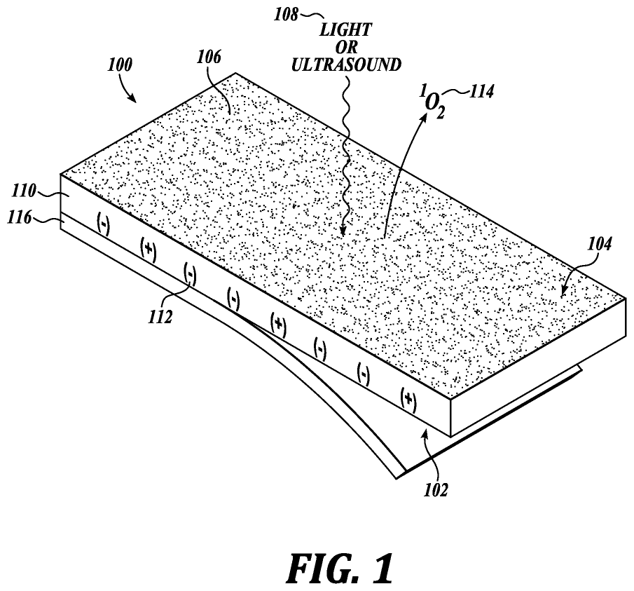

Referring to FIG. 1, an example of an antimicrobial film 100 includes a first surface 102 and a second surface 104 facing opposite from the first surface. Each of the first and second surfaces 102, 104 extends a width and length of the film 100, and a distance separating the first 102 and second 104 surfaces corresponds to a thickness of the film 100. The antimicrobial film 100 can include one or more material layers 110 between the first 102 and the second 104 surfaces of the film 100. The first surface 102 of the film 100 is adhesive. The second surface 104 of the film 104 emits singlet oxygen 114 when activated by light or ultrasound 108. In the antimicrobial film 100, the first surface 102 is adhesive through the presence of positive or negative charges 112 on the first surface 102. A material 110 that can form electrostatic charges 112 on the first surface 102 includes, but is not limited to, polyvinylidene chloride (PVDC), polyvinyl chloride, (PVC), polyethylene (PE), their derivatives, and combinations thereof.

In an example, the film 100 can include a first surface 102 facing in a first direction; a second surface 104 facing in a second direction opposite from the first surface. The first surface 102 and the second surface 104 extend along a width and a length of the film 100, wherein a distance separating the first surface and the second surface corresponds to a thickness of the film. One or more photosensitizers are arranged to emit localized singlet oxygen that is toxic to microbial particles incident on the second surface 104. The one or more photosensitizers are activated by one or more of light or ultrasound to generate the emitted singlet oxygen. In an example, the film 100 further comprises one or more material layers that are included between the first and second surfaces of the film, wherein the one or more material layers include the one or more photosensitizers. In an example, the first surface 102 of the film 100 is adhesive.

In one or more embodiments, any type of light source, including sunlight, ambient light, and/or artificial light, can be used that emits wavebands or wavelengths of light that are effectively absorbed by the photosensitizers to cause singlet oxygen generation. The illumination time and intensity of light needed for adequate antimicrobial disinfection of the film by a cloud of generated singlet oxygen may be determined empirically, experimentally, and/or derived from known data. In some embodiments, the light source can be comprised of light emitting diodes (LED), xenon lamps, fluorescent bulbs and tubes, incandescent light bulbs, electroluminescent devices, lasers, and the like, even including sunlight. Other known or contemplated light sources are not excluded in any fashion, and include all known wavelengths and wavebands known to lead to a photodynamic effect that generates singlet oxygen which is particular to the photosensitizer or combinations of different types and amounts of photosensitizers.

Additionally, although not shown, one or more embodiments of the antimicrobial film may include one or more portions of the antimicrobial film that are one or more of transparent, translucent or opaque to emitted light from one or more light sources.

Accordingly, in the antimicrobial film 100, the material layer 110 includes both the adhesive first surface 102 and the second surface 104 that emits singlet oxygen 114. The second surface 104 of the material layer 110 has one or more photosensitizers 106 incorporated thereon that can emit the singlet oxygen 114 as further described herein.

Optionally, a protective peel layer 116 can be adhered to the first surface 102 of the antimicrobial film 100 to prevent contamination of the first surface 102. When the antimicrobial film 100 is ready to be applied to an object or surface, the protective peel layer 116 is removed from the antimicrobial film 100. The protective peel layer 116 is not included in the thickness of the antimicrobial film 100. The protective peel layer 116 can be omitted, for example, when the antimicrobial film 100 is provided in a roll that can be unwound and cut to the length appropriate to the application. The protective peel layer 116 can be included, for example, when the antimicrobial film 100 is provided as a pre-cut individual item, or when the antimicrobial film 100 is provided in a distinctive shape. Further, a larger sheet can include the protective peel layer 116 onto which a plurality of pre-cut patches of the antimicrobial film 100 are adhered. The pre-cut patches can then be removed individually from the larger sheet.

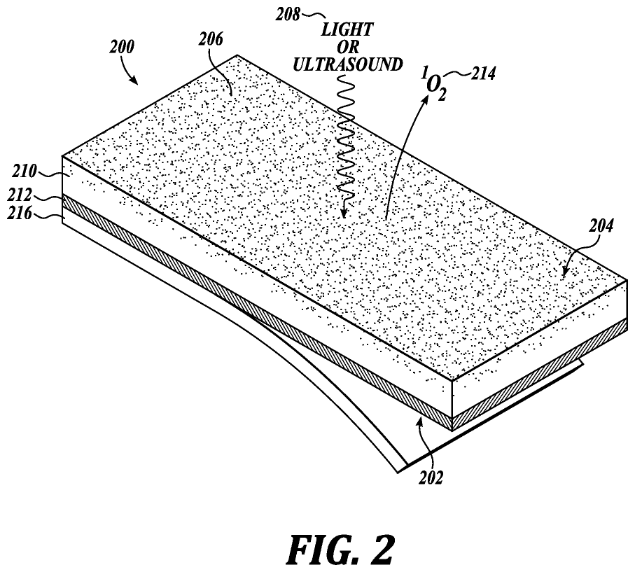

Referring to FIG. 2, an example of an antimicrobial film 200 includes a first surface 202 and a second surface 204 facing opposite from the first surface. Each of the first and second surfaces 202, 204 extends a width and length of the film 200, and a distance separating the first 202 and second 204 surfaces corresponds to a thickness of the film 200. The antimicrobial film 200 includes material layers 210 and 212 between the first 202 and the second 204 surfaces of the film 200. The first surface 202 of the film 200 is adhesive. The second surface 204 of the film 204 emits singlet oxygen 214 when activated by light or ultrasound 208. In the antimicrobial film 200, the first surface 202 is adhesive through the presence of a coating 212 of an adhesive composition. In one example, the first surface 202 uses a biocompatible adhesive for adhering to skin and other living tissues. The adhesive coating 212, can include but is not limited to, one or more adhesives selected from acrylates, silicones, hydrocolloid substances, and combinations. The adhesive coating 212 can be selected based on the intended application of the antimicrobial film 200. Accordingly, the antimicrobial film 200 comprises two material layers, an adhesive coating material layer 212 forms the first surface 212, and a second material layer 210 that has incorporated thereon one or more photosensitizers that emit the singlet oxygen 214. Examples of the second material layer 210 can include, but are not limited to, any polymer, plastic, cellulose, and other materials that can incorporate the photosensitizer compositions. The adhesive coating layer 212 is juxtaposed to the second material 210 in a non-separable manner.

Optionally, a protective peel layer 216 can be adhered to the first surface 202 of the antimicrobial film 200 to prevent contamination of the first surface 202. When the antimicrobial film 200 is ready to be applied to an object or surface, the protective peel layer 216 is removed from the antimicrobial film 200. The protective peel layer 216 is not included in the thickness of the antimicrobial film 200. The protective peel layer 216 can be omitted, for example, when the antimicrobial film 200 is provided in a roll that can be unwound and cut to the length appropriate to the application. The protective peel layer 216 can be included, for example, when the antimicrobial film 100 is provided as a pre-cut individual item, or when the antimicrobial film 200 is provided in a distinctive shape. Further, a larger sheet can include the protective peel layer 216 onto which a plurality of pre-cut patches of the antimicrobial film 200 are adhered. The pre-cut patches can then be removed individually from the larger sheet.

The antimicrobial films 100, 200 can have a thickness ranging from 1.0 micron to 5.0 millimeters (mm), though thinner or thicker embodiments are suitable for use. Film thicknesses can depend on the application. Application on smaller surface areas may use thinner films, while application on larger surfaces may require thicker films for strength. The thickness of the antimicrobial films 100 and 200 does not include the optional peel layer 106 and 206, respectively.

Antimicrobial films 100, 200, with and without the optional peel layer 106 and 206, can be flexible and some embodiments of the antimicrobial films 100, 200 can stretch in the length and width direction to conform to irregular surfaces, such as facial features. The width and length of the antimicrobial films 100 and 200 for application on facial features or wearable objects can vary, but generally can range from 1 mm to 10 mm and 1 cm to 10 cm, respectively.

Antimicrobial films 100, 200 can also be wound into rolls making their width and length much greater than just described for use on face or wearable objects. For application, the roll of antimicrobial film is unwound and cut to the length appropriate to the application. Larger rolls of antimicrobial films 100 and 200 can be used for covering large inanimate surfaces.

Both antimicrobial films 100 and 200 can be made from visible light-transparent materials to be less noticeable when worn on facial features or when applied to glass and other light transparent surfaces. That is, the material layer or layers between the first surface 102 and the second surface 202 of antimicrobial film 100 and the material layer or layers between the first surface 202 and 204 of antimicrobial film 200 are transparent to visible light.

The antimicrobial films 100, 200 incorporate at least one photosensitizer at least on the second surfaces 104 and 204, respectively. In antimicrobial film 100, the photosensitizer 106 can be incorporated in the material layer 110 which includes the second surface 104. In the antimicrobial film 200, the photosensitizer 206 can be incorporated in the material layer 210 which includes the second surface 204. Both material layer 110 of antimicrobial film 100 and material layer 210 of antimicrobial film 200 can use biocompatible polymers such as chitosan, various polyesters, polyurethanes, polyethylenes, alginates, gelatins, polyvinyl compounds, as the material layer of the second surface 104, 204.

In some examples, the antimicrobial films 100 and 200 can include additional material layers (not illustrated) that do not form either the first or second surfaces of the antimicrobial films. Such additional material layers can add functionality, such as to function as a vapor and moisture barrier or to provide further strength or may incorporate other beneficial compositions.

A photosensitizer is a compound that can generate at least singlet oxygen in response to light provided at particular wavebands or wavelengths and ultrasound provided at particular frequencies and for a particular duration. Singlet oxygen is known by the chemical formula, ¹O₂. Photosensitizers include, but not limited to, all types of methylene blue derivatives and methylene blue itself, xanthene dyes and derivatives, chlorophyll derivatives, tetrapyrrole structures, porphyrins, chlorins, bacteriochlorins, phthalocyanines, texaphyrins, prodrugs such as aminolevulinic acids, phenothiaziniums, squaraine, boron compounds, various transition metal complexes, hypericin, riboflavin, curcumin, titanium dioxide, psoralens, tetracyclines, flavins such as riboflavin, riboflavin derivatives, erythrosine, erythrosine derivatives, and the like. Preferred photosensitizers are a combination of ones that are generally recognized as safe, and that are capable of absorbing light over a wide spectral range, such as a combination of erythrosine, methylene blue, and riboflavin.

Methods for incorporating into and adding one or more photosensitizers to material layers 110 and 210 include dip coating, spray coating, solvent casting, spin coating, photo crosslinking, vapor deposition, and the like. The incorporation of the one or more photosensitizers onto the films can occur before or after the application of the film onto the surface of interest for disinfection. Further, in some examples, films once applied can be recharged with one or more photosensitizers after the lapse of a period of time deemed to exhaust the photosensitizer activity. Antimicrobial films 100 and 200 may also incorporate material layers of porous materials and foams for absorption of photosensitizers and for breathability for use as the second surface 104, 204.

Methods of creating films include self-assembled monolayers, surface-tethered polymers (polymer brushes), or multilayer coatings based on layer-by-layer assembly, using 3-D printing techniques as an example. Hydrophilic films can be based on polyethylene glycol, tertiary amine oxides, polyacrylamides, various methylated esterified compounds, and cross linked photoinitiators such as benzophenone, which can be used to incorporate one or more hydrophilic photosensitizers. Hyperbranched polymers with hydrophilic groups such as dendritic polyglycerols can also be used to retain one or more photosensitizers in a film. Polyelectrolyte multilayer films may incorporate phosphorylcholines. Polyurethane films containing hyaluronic acid which can incorporate one or more photosensitizers can also be manufactured into antimicrobial films. Biocompatible carbon nanotube polymer composites can be electrically charged, with the charge aiding in viral capture and retention for photodynamic disinfection. A carrier such as chitosan can be used to adhere one or more photosensitizers onto a film, or be used to create a film itself.

Biocompatible adhesives, comprised of synthetic rubber compounds, acrylates, or silicones can be applied to the surface of the antimicrobial film, which may be covered with the non-stick peel-away layer made of plastic or paper and is removed prior to cutaneous or intraoral manual application.

The concentration of at least one photosensitizer to be incorporated or applied to the films to produce the antimicrobial films of this disclosure may be determined experimentally by incorporating or applying different concentrations of the photosensitizer to the film, applying a bacterial, fungal, or viral contaminant to the film surface to simulate airborne and contact contamination of a surface, and then delivering light. Light emitted by light sources commonly found in the medical environment, of different intensities, wavelengths, and wavebands, at different distances from the film, at different fluence rates and illumination time periods can be delivered to the film surface. The antimicrobial film can be wrapped around test phantoms and objects which simulate those found in the medical environment, in order to test the photoinactivation effect when objects in the medical environment are exposed to light from different angles and to test the various parameters of wavelength, waveband, illumination time, intensity, fluence rates and distance of the light source from the object. Swabs or film samples are compared after illumination using know laboratory techniques in order to determine microbicidal activity on the film surface, which informs the best photosensitizer and photosensitizer concentration to incorporate into the films, and the frequency at which the film needs to removed and replaced, or recharged.

The antimicrobial films 100, 200 can contain an effective amount of one or more photosensitizers 106, 206. As an example, the effective concentration range of the one or more photosensitizers can be from 0.05 μM to 1000 μM, and any included range, such as concentration ranges of 0.05 μM to 0.5 μM, 0.5 μM to 5 μM , 5 μM to 50 μM, 50 μM to 100 μM, 100 μM to 500 μM , 500 μM to 1000 μM.

Once applied to a surface, the antimicrobial films 100 and 200 with the incorporated photosensitizers can be exposed to light or ultrasound to generate antimicrobial singlet oxygen in a localized fashion. The singlet oxygen can collect in a zone in proximity to the antimicrobial films 100 and 200 to provide protection from microbial particles, e.g., viruses and pathogens. An effective amount of light or ultrasound 108, 208 absorbed by the photosensitizers 106, 206 on the surfaces of the films 100, 200 creates localized singlet oxygen and other radical species which are toxic to microbial particles. Light includes any ambient indoor or outdoor light including sunlight. Any type of light source including sunlight, ambient light, and/or artificial light, can be used that emits the proper wavebands or wavelengths of light that are effectively absorbed by the photosensitizers to cause singlet oxygen generation. The illumination time and intensity of light needed for adequate generation of singlet oxygen may be determined empirically, experimentally, and/or derived from known data. In some embodiments, the light source can be comprised of light emitting diodes (LED), xenon lamps, fluorescent bulbs and tubes, incandescent light bulbs, electroluminescent devices, lasers, and the like, even including sunlight. Other known or contemplated light sources are not excluded in any fashion, and include all known wavelengths and wavebands known to lead to a photodynamic effect that generates singlet oxygen which is particular to the photosensitizer or combinations of different types and amounts of photosensitizers.

In one example, an effective amount of light corresponds to an exposure time that can range from 1 second to 2 hours, and the lux (lumen per square meter) can range from 10 to 50,000. In one example, a preferred exposure time is from 1 minute to 1 hour and a lux range from 100 to 10,000. In one embodiment, the most preferred exposure time is from 5 minutes to 30 minutes, and a lux range from 100 to 10,000.

An effective amount of ultrasound can be in the range of, for example, from 5 kHz to 20 kHz, from 20 kHz to 100 kHz, from 100 kHz to 1 MHz, from 1 MHz to 30 MHz with a power density ranging from 0.001 to 1.0 W/cm², from 1.0 to 10 W/cm², and from 10 to 12 W/cm². Duration of exposure can range from 1 second to 60 minutes, from 60 minutes to 10 hours. The duty cycle (the “on” time of the ultrasound pulses) can range from 0.5% to 99.9% of the treatment time.

In some examples, the antimicrobial films 100 and 200 can be adhered directly on the exterior of facial skin, on the interior of the oral cavity, or on any wearable object that is configured to be placed on the head or face, and also for use on personal protective equipment.

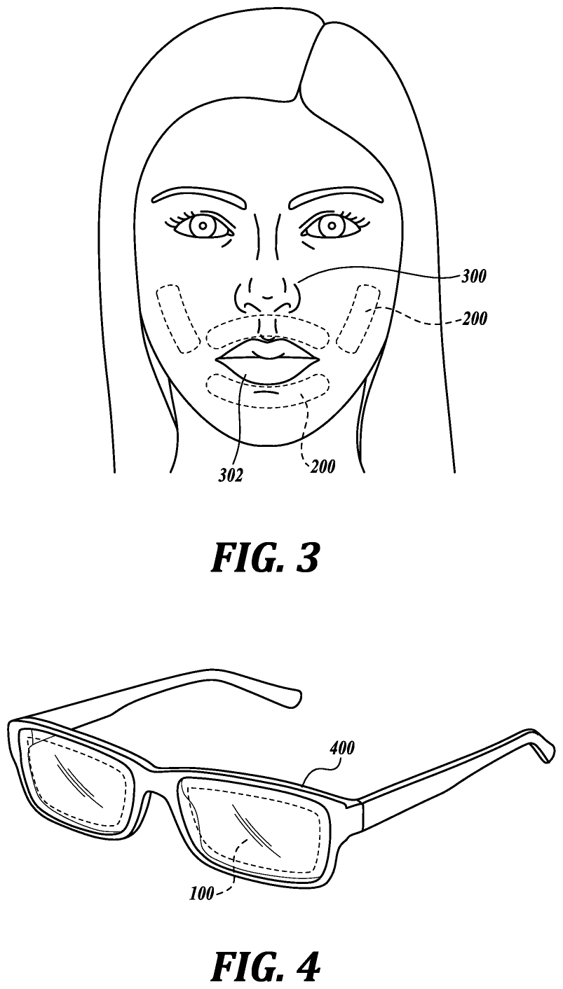

FIG. 3 illustrates an antimicrobial film, such as antimicrobial film 200, with a biocompatible adhesive coating 212, is positioned on and adheres to the exterior surfaces of facial skin. Antimicrobial film 200 with biocompatible adhesive 212 can be placed under the nose 300 and/or the facial skin around the lips 302 on the exterior. Additionally, in one example, the antimicrobial film 200 has an adhesive composition 212 configured to be adhered to teeth. In one example, the antimicrobial film 200 has an adhesive composition 212 configured to be adhered to the oral cavity interior, in particular to the mucous membranes lining the interior of the oral cavity such as the inside of the cheek. For applications including facial features and oral cavity interior, the antimicrobial film 200 can be pre-cut in a distinctive pattern designed for use to a particular location.

FIG. 4 illustrates a pair of glasses 400 having an antimicrobial film, such as film 100, adhered to the inside and/or outside of the lenses. Here, a light-transparent film 100 is used for adhering to glass surfaces. Further, the material of the film 100 can adhere without the addition of an adhesive composition. Additional antimicrobial films 100 can optionally be placed on the nose bridge, other parts of the frame, and the temples. Optional side shields and/or a nasal shield can be attached to the glasses' frame and temples in order to increase the surface area over which antimicrobial film 100 or 200 can be incorporated, enabling light activated singlet oxygen generation in a larger volume of air in front of and around the user's face.

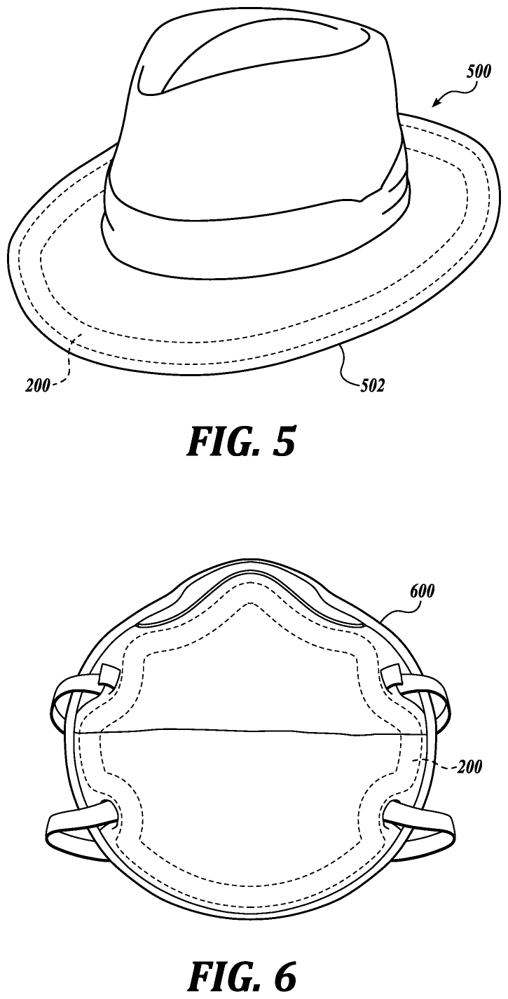

FIG. 5 illustrates a hat 500 has the antimicrobial film 200 adhered to the underside of the brim 502. The antimicrobial film 200 can be provided with a range of adhesive compositions depending on the application. Attachment to the underside of a hat brim can generate a shower of singlet oxygen molecules when activated by light, in front of the user's face, which can then inactivate viral laden aerosols and droplets prior to reaching the eyes, nose, or mouth of the user. In relatively still air conditions, such as in indoor settings, the risk of inhalational viral exposure is reduced in this manner.

FIG. 6 illustrates a protective mask 600, such as an N-95 mask, that has an antimicrobial film 200 adhered to the exterior of the mask 600 and around the perimeter edges of the mask 600. The antimicrobial film 200 offers additional protection beyond what is provided by the mask 600 alone. The placement of the antimicrobial film 200 around the perimeter of the mask 600 can be advantageous, since during inhalation any air that may bypass the mask material will be inhaled through the gaps between the mask and skin, and in so doing, the air will pass in close proximity to the antimicrobial film 200 and the zone of singlet oxygen generated therefrom.

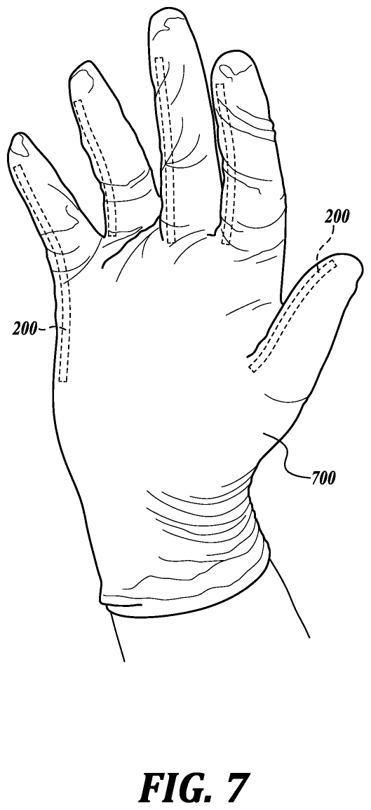

FIG. 7 illustrates a protective glove 700 that has an antimicrobial film 200 adhered to the exterior of the glove 700, such as along the exterior of the fingers. The placement of the antimicrobial film 200 on the exterior of the fingers can be advantageous, since any microbial particles, e.g., viruses and pathogens, that come into contact with the glove will likely occur at the fingers through touching, the microbial particles will then be in close proximity to the antimicrobial film 200 and the zone of singlet oxygen generated therefrom.

FIG. 7 illustrates a protective glove 700 that has an antimicrobial film 200 adhered to the exterior of the glove 700, such as along the exterior of the fingers. The antimicrobial film 200 offers additional protection beyond what is provided by the glove 700 alone. The placement of the antimicrobial film 200 on the exterior of the fingers can be advantageous, since any microbial particles that are transferred to the glove 700 through touch will likely occur at the fingers. The microbial particles will then be in close proximity to the antimicrobial film 200 and the zone of singlet oxygen generated therefrom.

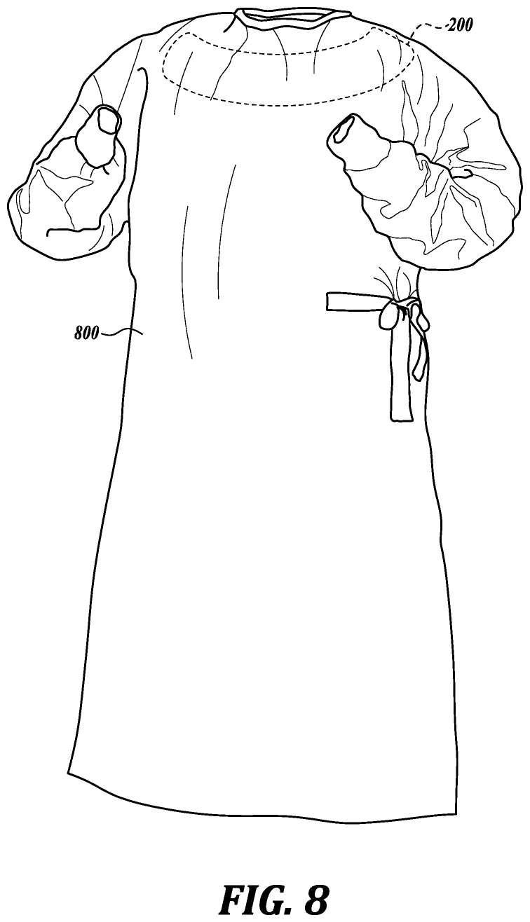

FIG. 8 illustrates a protective gown 800 that has an antimicrobial film 200 adhered to the exterior and below the collar. The antimicrobial film 200 offers additional protection beyond what is provided by the gown 800 alone. The placement of the antimicrobial film 200 on the exterior of the gown 800 and below the collar can be advantageous, since the collar region can afford protection from microbial particles, e.g., viruses and pathogens, that are in proximity to the face and that may be inhaled by the wearer. Additional antimicrobial films 200 can be adhered to the gown 800 in places that are high-touch areas, for example, at the wrists, the abdomen, etc.

Example uses of antimicrobial films 100 and 200 also include use for medical equipment and the general medical environment. Antimicrobial films 100 and 200 enabling photodynamic action can lead to disinfection of surface adherent microbial particles on the surface of the film, external to the object surface, and not in direct contact with the object surface the film adheres to.

In one example, antimicrobial film 100 clings readily to smooth surfaces and can readily be removed and replaced or recharged as needed. The antimicrobial film 100 can be supplied in rolls or sheets and protected from light during storage and shipping in light opaque containers.

In one example, antimicrobial film 200 is made from a material that is permeable to air and is impregnated with at least one photosensitizer on the second surface 204 and is provided with an adhesive on the opposite first surface 202. The antimicrobial film 200 can be manufactured in rolls or sheets, and optionally utilizes intervening peel material to prevent self-adhesion. The sheets and/or rolls are cut to size and the antimicrobial film 200 is applied adhesive side down, against a mask, N95 respirator, or other PPE outer surface, with the opposite surface incorporating at least one photosensitizer in order to provide effective photodynamic inactivation when activated by ambient or discrete light sources against microbial particles which may come in contact with the outer surface of the mask, N95 respirator, or PPE.

In some examples, breathable films that feature an adhesive side include, but are not limited to tape manufactured as 3M Medical Transpore Tape™, breathable adhesive urethane films from Medco Coated Products, and breathable sealing films with an adhesive side from Thomas Scientific, or other nanocomposite material. Such existing films can be converted into antimicrobial films by the incorporation of photosensitizers as disclosed herein.

In some examples, the pore size and pore spacing of breathable antimicrobial films to permit free and comfortable air passage, but prevent viral or other pathogen penetration, is tested and optimized for mass manufacture, as is the optimal photosensitizer concentration on the film surface that reliably inactivates adherent microbial particles. New masks, N95 respirators, or PPE product may be manufactured including additional surface area to maintain adequate breathable air flow. The mask, N95 respirator, or PPE material can be protected from degradation due to the photodynamic action.

Yet another embodiment contemplates a lip balm formulation containing at least one photosensitizer which is applied to the lip surfaces, and provides for singlet oxygen generation when exposed to ambient light. The photosensitizer lip balm is supplied in a container similar to those used to contain lipstick and lipgloss.

In one example, singlet oxygen 114, 214 can be generated from antimicrobial films 100, 200 by directing light onto the second surfaces 104, 204 through an optical fiber coupled to a light source that may include at least one light emitting diode (LED). The LED or other light source is located external to the oral cavity, for example worn behind the ear, as in a hearing aid, and is powered by a rechargeable battery. The distal end of the optical fiber is positioned within the user's mouth.

In another example of a light source, at least one LED is positioned within the user's oral cavity and is powered by an external battery connected to lead wires, or alternatively, powered by an inductive coupling system which utilizes an external radiofrequency coil emitter which charges a tiny coil connected to the LED. An external battery can be worn behind the ear as is done for some types of hearing aids. The inductive coupling system uses a radiofrequency coil system to charge the LED device, or to supply the LED device with continuous electrical current. In one example, the LED device is attached to the user's teeth, in a fashion similar to a mouth guard.

In another example, a light source, at least one LED which is positioned within the user's oral cavity and is powered by a piezoelectric device.

In another example, an external ultrasound transducer aimed at the intraoral antimicrobial film 200 of FIG. 3 can be used to activate at least one photosensitizer non-invasively.

In another example, a small light opaque reservoir filled with a photosensitizing solution which is reversibly attached using bandage type adhesives, such as acrylates, is located unobtrusively under the chin. The reservoir is single-use or refillable. The reservoir is attached to a material in the form of a string that wicks the photosensitizing solution by way of capillary action to an antimicrobial film. The string like wicking material can be comprised of known polyesters, polypropylenes, or other materials such as wools, nylon, rayon, and even bamboo, when processed into tiny tubes. The wicking action serves to continually replenish the photosensitizer solution, enabling continuous, long-term generation of antimicrobial, protective singlet oxygen. The string can be reversibly and comfortably attached to the user's face, in particular around the nasal area, and around the oral cavity, and in the peri-orbital area, where viral penetration is apt to occur.

In another example, a small light opaque reservoir and wick is located behind the ear, similar to a hearing aid. Other optional locations for the light opaque reservoir and wick include as an attachment to a hat, or garments worn by the user.

In another example, antimicrobial films can be applied to and wrapped around high touch hospital room areas such as bed rails, tray tables, supply carts, monitoring screens, IV poles and pumps, vitals machines, wall shelves, handles and knobs, bathroom fixtures, in-room computers, and the like. During and after an infected patient has been cared for, and the room must be disinfected, the film is removed, and a fresh film applied. The ambient room light optionally augmented by other dedicated light sources is sufficient to provide for continuous disinfection of high touch surfaces, which reduces the risk to health care workers caring for the infected patient.

In another example, the antimicrobial films can be used where an outbreak of microbial particles, e.g., a virus or any other pathogen occurs on a cruise ship leading to confinement of infected or possibly infected persons in cabin rooms. The antimicrobial film is applied to furniture, bathroom fixtures, walls, ceiling, floor, and any other amenable surfaces. The photo-disinfection process occurs after the room is exposed to light. Surfaces in shadow can be exposed to aimed light such as from a lamp. The cleaning crew is able to change the film as needed. The room can remain occupied by the infected patient which is an advantage in reducing transmission of the microbial particles, compared with other decontamination techniques using toxic vapors as an example, where the room must be vacated.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments and examples of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

While illustrative embodiments have been illustrated and described, it will be appreciated that various changes can be made therein without departing from the spirit and scope of the invention.

The embodiments of the invention in which an exclusive property or privilege is claimed are defined as follows:

1. An antimicrobial film, comprising:
a first surface facing in a first direction;
a second surface facing in a second direction opposite from the first surface, wherein the first surface and the second surface extend along a width and a length of the film, and wherein a distance separating the first surface and the second surface corresponds to a thickness of the film;
one or more material layers are included between the first and the second surfaces of the film;
wherein the first surface of the film is adhesive; and
wherein the second surface of the film emits singlet oxygen that is toxic to microbial particles incident on the second surface when activated by light or ultrasound.

2. The antimicrobial film of claim 1, comprising one or more photosensitizers in or on at least one material layer.

3. The antimicrobial film of claim 2, wherein the one or more photosensitizers include one or more of erythrosine, riboflavin, or methylene blue.

4. The antimicrobial film of claim 1, wherein the first surface includes electrostatic charges as an adhesive.

5. The antimicrobial film of claim 4, wherein the first surface is on a first material layer comprising polyvinylidene chloride, polyvinyl chloride, or polyethylene.

6. The antimicrobial film of claim 5, further comprising a second material layer juxtaposed on the first material layer, wherein the second material layer includes a polymer, plastic, cellulose, and the second material layer is the second surface of the film.

7. The antimicrobial film of claim 1, wherein the first side includes an adhesive coating as a first material layer.

8. The antimicrobial film of claim 7, wherein the adhesive coating includes one or more adhesives selected from acrylates, silicones, and hydrocolloid substances.

9. The antimicrobial film of claim 7, further comprising a polymer, plastic, or cellulose as a second material layer, the adhesive coating is juxtaposed on the second material layer.

9. The antimicrobial film of claim 9, wherein the second material layer includes one or more photosensitizers, and the second material layer includes the second surface of the film.

11. The antimicrobial film of claim 1, wherein more than one material layers between the first and second surfaces are non-separable.

12. The antimicrobial film of claim 1, further comprising a peel layer juxtaposed on the first surface, the peel layer is separable from the film to expose the adhesive first surface.

13. The antimicrobial film of claim 1, wherein the thickness of the film is from 1.0 micron to 10 mm.

14. The antimicrobial film of claim 1, wherein the thickness of the film is from 1 mm to 10 mm.

15. The antimicrobial film of claim 1, wherein the second surface comprises an absorbent material, and the one or more photosensitizers are absorbed in the absorbent material.

16. The antimicrobial film of claim 15, wherein the absorbent material is rechargeable with the one or more photosensitizers.

17. The antimicrobial film of claim 1, wherein the antimicrobial film further comprises one or more portions that are transparent, translucent, or opaque to visible light.

18. A wearable object for the face or head, comprising:
a wearable object with means for holding the wearable object to the face or head; and
the antimicrobial film of claim 1 is adhered to the wearable object.

19. The wearable object of claim 18, wherein the wearable object is a hat or glasses.

20. A method of inactivating pathogens in an oral cavity, comprising:
applying the antimicrobial film of claim 1 to a surface inside of an oral cavity; and
applying light or ultrasound energy directed at the second surface of the antimicrobial film to cause the emission of singlet oxygen from the film in the oral cavity.

21. A film, comprising:
a first surface facing in a first direction;
a second surface facing in a second direction opposite from the first surface, wherein the first surface and the second surface extend along a width and a length of the film, and wherein a distance separating the first surface and the second surface corresponds to a thickness of the film;
one or more photosensitizers that are arranged to emit localized singlet oxygen that is toxic to microbial particles incident on the second surface, wherein the one or more photosensitizers are activated by one or more of light or ultrasound to generate the emitted singlet oxygen.

22. The film of claim 21, further comprising one or more material layers that are included between the first and second surfaces of the film, wherein the one or more material layers include the one or more photosensitizers.

23. The film of claim 21, wherein the first surface of the film is adhesive.
